(19) United States
(12) Patent Application Publication
(10) Pub. No.: US 2017/0191982 A1
(43) Pub. Date: Jul. 6, 2017

(54) Constriction-Expansion Blood Plasma Separation

(71) Applicant: Massachusetts Institute of Technology, Cambridge, MA (US)

(72) Inventors: Tatyana A. Shatova, Cambridge, MA (US); Klavs F. Jensen, Lexington, MA (US); Marissa R. Engle, Sunnyvale, CA (US)

(21) Appl. No.: 15/399,285

(22) Filed: Jan. 5, 2017

Related U.S. Application Data
(60) Provisional application No. 62/275,429, filed on Jan. 6, 2016.

(51) Int. Cl. G01N 33/49; B01L 3/00; A61M 1/34

(52) U.S. Cl. CPC G01N 33/491; A61M 1/3496; B01L 3/502715; B01L 3/502753; B01L 3/502761; B01L 3/50273; B01L 2300/0864; B01L 2300/12; B01L 2300/0627; B01L 2400/0478; B01L 2200/0647

(57) ABSTRACT

A portable, microfluidic blood plasma separation device is presented featuring a constriction-expansion design, which can produce up to about 100% purity for undiluted blood at least about 9% yield. This level of purity represents an improvement of at least one order of magnitude with increased yield compared to that achieved previously using passive separation. The system features high flow rates, 5-30 μL/min plasma collection, with minimal clogging and biofouling. The simple, portable blood plasma separation design can be hand-driven and can easily be incorporated with microfluidic or laboratory scale diagnostic assays. The separation system can be used in conjunction with portable analyte detection tests at concentrations well below clinical relevancy for undiluted whole blood.

### RELATED APPLICATION

This application claims the benefit of U.S. Provisional Application No. 62/275,429, filed on Jan. 6, 2016. The entire teachings of the above application are incorporated herein by reference.

### GOVERNMENT SUPPORT

This research was supported (in part) by the U.S. Army Research Office under contract W911NF-13-D-0001 (KFJ). The government has certain rights in this invention.

### FIELD OF THE INVENTION

A portable, microfluidic blood plasma separation device is presented featuring a constriction-expansion design, which can produce up to about 100% purity for undiluted blood at least about 9% yield. This level of purity represents an improvement of at least one order of magnitude with increased yield compared to that achieved previously using passive separation. The system features high flow rates, 5-30 μL/min plasma collection, with minimal clogging and biofouling. The simple, portable blood plasma separation design can be hand-driven and can easily be incorporated with microfluidic or laboratory scale diagnostic assays. The separation system can be used in conjunction with portable analyte detection tests at concentrations well below clinical relevancy for undiluted whole blood.

### BACKGROUND OF THE INVENTION

Blood plasma and serum are the most widely used biological fluids in biomedical and diagnostic microfluidic and laboratory applications.¹ Blood contains a variety of biomarkers that can be used for detection of immune system response to foreign objects, traumas, inflammation during cardiac surgeries, presence of foreign biomarkers due to infectious diseases, as well as for long-term management of autoimmune disease and cancer.²⁻⁵ However, a high concentration of cells in blood (10⁹ cells per mL) often limits point-of-care (POC) diagnostics to pretreated blood or serum. The laboratory standard for blood plasma isolation is centrifugation, which requires laboratory equipment, training, a higher blood volume compared to microfluidics, and does not allow for easy incorporation with microfluidic analysis.⁶ Therefore biological sample handling and plasma separation remains a limiting component of microfluidic biodetection and POC diagnostics.²

### SUMMARY OF THE INVENTION

A portable, microfluidic blood plasma separation device is presented featuring a constriction-expansion design, which can produce up to about 100% purity for undiluted blood at least about 9% yield. This level of purity represents an improvement of at least one order of magnitude with increased yield compared to that achieved previously using passive separation. The system features high flow rates, 5-30 μL/min plasma collection, with minimal clogging and biofouling. The simple, portable blood plasma separation design can be hand-driven and can easily be incorporated with microfluidic or laboratory scale diagnostic assays. The separation system can be used in conjunction with portable analyte detection tests at concentrations well below clinical relevancy for undiluted whole blood.

In order to minimize the complexity of the system, and to extend the application of the construct to any type of microfluidic detection system, we focused on passive blood separation approaches that do not require the use of an external field. Active separation, which includes acoustic, electric, and magnetic approaches have been described elsewhere.^{1,2} The simplest of the passive separation methods is sedimentation, which takes advantage of gravity and the difference in density between plasma and blood cells (1030 kg/m³ and 1050-1100 kg/m³, respectively).^{7,8} Sedimentation requires long time periods and therefore cannot be the sole driver for blood separation. Despite this limitation, it is occasionally used for blood separation in combination with other methods, such as dead-end filtration, which features a filter perpendicular to flow, or cross-flow filtration, which features a filter(s) that is parallel to the channel and the flow direction.^{9,10} However, the high number of red blood cells present and the extreme deformability of these small discoid cells (red blood cells are approximately 2 μm thick, and 8 μm in diameter), lead to the buildup of a fouling layer on the filter in both methods and thus short device lifetimes. Therefore, other components such as a wash buffer, pulsating flow, or most recently a balance of sedimentation and flow rate have been incorporated into these devices to improve the collected fraction volumes.^{11,12} Instead of fabricating or incorporating filters into a channel, which can increase complexity or lead to leaks around the edges, other efforts have incorporated obstacles to deviate blood cells from plasma collection. Unfortunately, as in filtration, the high cell fraction in human blood as well as the extreme deformability and discoid shape of red blood cells limit these applications in blood separation. Also, similarly to dead-end filtration, most of the obstacle-deviated flow devices feature complex fabrication steps requiring exact feature sizes, high dilution numbers, slow flow rates, and are prone to nonspecific binding.^{2,13}

Another passive separation method is based on microfluidic channel hemodynamics that incorporates inertial, viscous, cell-cell interaction, cell-protein interaction, and other forces stemming from the channel geometry and flow for plasma separation. In these systems, both rigid and deformable cells have been found to migrate across stream-lines based on shear gradient lift and wall lift due to the asymmetry of the fluid profile around the cell.¹⁴⁻¹⁷ In blood, this creates a cell-free layer, which also depends on the concentration of cells, cell-cell interactions, and cell-protein interactions.¹⁸ This phenomenon has also been studied for plasma skimming approaches, and has been termed the Zweifach-Fung bifurcation effect.¹⁹⁻²⁴

This application focuses on inertially-dominant systems which allow for high throughput of plasma to enable fast sensor readout. Inertial cell sorting in microfluidic devices is often applied for dilute solutions, which eliminate any particle or protein interactions, and allow for separation based on size.¹⁶ This approach has also been applied to trapping rare cells through the use of rapid expansions and contractions in the channel.^{14,17} The geometry creates vortices within the expansion and can trap cells that have contrasting physical parameters from the most concentrated particles. The method takes advantage of the fact that the majority of cells do not follow the streamlines into the expansion traps. The abrupt expansion approach was applied for plasma separation and showed a significant improvement over literature plasma skimming values due to a temporary increase in the plasma layer near the channel expansion, despite the cell vortices for certain flow regimes.¹⁸

The approach described in herein focuses on an optimized microfluidic channel design that incorporates a constriction followed by a gradual channel expansion, which minimizes the cell-trapping vortices while still enhancing the cell-free layer volume. These improvements increase the purity of the plasma collected from both diluted and undiluted whole blood beyond what has been demonstrated to date. The lack of complexity allows for the incorporation with a variety of microfluidic sensor constructs and minimizes design features that can promote biofouling.

A purpose of this work is to describe an innovative, portable design that can separate blood plasma, for a range of blood dilution values, without the need for external fields or external equipment. We also verified that the plasma separation system could be combined with downstream point-of-care diagnostic testing using a paper-based colorimetric test that requires a cell-free sample. A recently reported paper-based test used polymerization-based signal amplification (PBA) to provide robust detection of Plasmodium falciparum histidine-rich protein 2 (PfHRP2) in small volumes of serum (10 μL per test).²⁵ PfHRP2 is a soluble protein that is released into the blood stream of malarial patients and is used as a biomarker in commercial rapid diagnostic tests.²⁶ The paper-based PBA test could be performed in air, required less than 100 seconds for the photopolymerization reaction in visible light and used inexpensive reagents^{25,27,28}, making it suitable for low-cost diagnostic applications. The analytical capabilities of this approach have not yet been established for whole blood samples. Pairing this test with a device that rapidly prepares plasma from small volumes of whole blood in low-infrastructure settings represents a critical step towards taking diagnostic tests, which require whole blood samples, away from the laboratory settings to where they are needed the most.

The invention includes a cell separation tool, and particularly a blood plasma collection tool, comprising an inlet channel; an expansion channel in fluid communication with the inlet channel, a width of a proximal end of the expansion channel being approximately equal to a width of the inlet channel, and a width of a distal end of the expansion channel being larger than the width of the inlet channel; at least one side channel in fluid communication with the expansion channel and configured to divert fluid flow from the expansion channel; and an outlet channel in fluid communication with the expansion channel. Preferably, the inlet channel has a first fluid flow region and the expansion channel has a second flow region with a different fluid flow pattern than the first fluid flow region. Preferably, the expansion channel has an angle of expansion effective to achieve a cell free layer in the expansion channel proximal to the at least one side channel and, optionally, an angle of expansion effective to produce similar cell separation at a plurality of flow rates. Typically, the expansion channel has an angle of expansion between about 5 degrees and about 20 degrees, preferably between about 5 and 10 degrees. In general, each channel is a microchannel. For example, the width of the inlet channel is less than about 200 μm, preferably less than 100 μm or less than 50 μm, and/or the width of the outlet channel is less than about 400 μm, preferably less than 200 μm, and/or the width of the at least one side channel is less than 200 μm, preferably less than 100 μm or less than 50 μm. Additionally or alternatively, the height of each channel are each, independently, less than about 400 μm, preferably less than about 200 μm. Preferably, the expansion channel and each side channel are in fluid communication with a collection reservoir and/or the inlet channel is in fluid communication with an inlet reservoir.

The invention also includes methods of using the tools of the invention, particularly for separating cells from a fluid, such as in collecting plasma from blood.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A. View (a) Schematic of the experimental setup (not to scale). View (b) Schematic of the blood plasma separation device design; device dimensions in mm, Exp < is the expansion angle, defined as deviation from the horizontal axis. View (c) An example microscope image of 0.1× blood cell free development in the expansion region of the device.

FIG. 1B View (A) illustrates the device angle of expansion. View (B) is a microscopic image of an example of a separation device. The dark spots are blood cells while the clear region near the wall is the cell free layer or plasma.

FIG. 2. Isolate separation ratio vs. yield for 5-20° expansions for (a) 0.1× blood volume fraction, (b) 0.3× blood volume fraction, and (c) 1× blood volume fraction or undiluted whole blood. The horizontal line at 100 isolate separation ratio indicates an equivalent of 99% purity. The expansion angle of the blood separation devices is defined in the figure legend. Each point represents one experiment. Yield is defined as mass fraction of the total, maximum yield that is achievable is between 0.4-0.6 for undiluted blood.

FIG. 3. Separation ratio vs. yield for the 5° expansion design, comparing syringe pump-driven and hand-driven results for 1×, 0.3×, and 0.1× blood volume fractions (1×B., 0.3×B. and 0.1×B., respectively). The horizontal line at 100 isolate separation ratio indicates an equivalent of 99% purity. Each point represents one experiment. Yield is defined as mass fraction of the total, therefore maximum yield that is achievable is between 0.4-0.6 for undiluted blood.

FIG. 4. Colorimetric detection of PfHRP2 (0-130 nM) in whole blood. Visual LOD, the concentration of PfHRP2 where all replicates generate a result that is easily visible to the unaided eye, was 7.5 nM and falls well below the clinically relevant concentration of 28 nM identified in a patient cohort.³⁴

FIG. 5. Effect of purity of plasma sample on background color of paper tests. 10 μL samples of pure plasma (prepared via centrifugation), 100 isolate separation ratio (or 99% purity previously shown in the literature), and 2,800 isolate separation ratio (or 99.96% purity achieved by the device described in this manuscript) were applied to paper surfaces for one hour in a humid chamber. (a) Representative images of paper surfaces containing 10 μL sample (Wet), blotted sample without washing (0 Wash), with one wash of 20 uL solution of 1×PBS (1 Wash), and two washes each of 20 μL solution of 1×PBS (2 Wash) are shown. (b) Absolute difference in the mean colorimetric intensity of paper surfaces incubated with 99% and 99.96% pure plasma samples compared to surfaces incubated with pure plasma. The error bars represent standard deviation (n=6). The plasma samples with 99.96% purity are visually and quantitatively similar to the pure plasma samples and a single buffer wash is sufficient to remove any background color from these surfaces. The samples with 99% purity produce a visible red hue on the paper surface even after two washes; this background color can lower the visual contrast and interfere with accurate interpretation of positive test results.

### DETAILED DESCRIPTION OF THE INVENTION

The invention includes tools for separating cells from a fluid and methods of use. In particular, the invention includes a cell separation tool comprising: an inlet channel; an expansion channel in fluid communication with the inlet channel, a width of a proximal end of the expansion channel being approximately equal to a width of the inlet channel, and a width of a distal end of the expansion channel being larger than the width of the inlet channel; at least one side channel in fluid communication with the expansion channel and configured to divert fluid flow from the expansion channel; and an outlet channel in fluid communication with the expansion channel. In preferred embodiments, the tool is integrated onto microfluidic chip.

Preferably, one or more, such as each, channel in the tool are microchannels, such as microchannels having dimensions, less than 1 mm. Conveniently, each channel is defined by two opposing walls, a floor or bottom and a top or ceiling. The opposing walls can define the width of the channels. Preferably, the width of the inlet channel is less than about 200 μm, preferably less than 100 μm or less than 50 μm, and/or the width of the outlet channel is less than about 400 μm, preferably less than 200 μm, and/or the width of the at least one side channel is less than 200 μm, preferably less than 100 μm or less than 50 μm. Of course, alternative geometries can be designed as well, including one or more channels having a circular, elliptical or trapezoidal cross section can be employed as well. Where the channel is closed, the height of the channel can be defined by the bottom (floor) and top (ceiling) surfaces. Otherwise the height of the channel can be defined by the height of the walls. Preferably, the height of the inlet channel, the expansion channel, the at least one side channel and the outlet channel are each, independently, less than about 400 μm, preferably less than about 200 μm, such as about 100 μm. In a preferred embodiment, the height of each channel is the same.

The relative width and height of each channel can affect the fluid flow, including the creation of vortices near the channel surfaces and walls. The inlet channel intersects the expansion channel at the expansion channel proximal end. At the junction, or point of intersection, the width of the inlet channel is preferably the same as the width of the proximal end of the expansion channel. Upstream of the junction, or point of intersection, the inlet channel preferably has consistent dimensions, such as the same width along the length of the inlet channel. Along the length of the expansion channel, the expansion channel expands from the junction or point of intersection with the inlet channel to the junction or point of intersection with the outlet channel. At the junction or point of intersection with the outlet channel, the outlet channel preferably has a width equal to the distal end of the expansion channel. Additionally, or alternatively, the width of the at least one side channel at its point of communication with the expansion channel is less than or equal to the width of the inlet channel. Preferably, the width of each side channel can increase along the length of the channel, thereby increasing yields of cell-free fluid, or plasma.

As the width of the outlet channel is greater than the width of the inlet channel, at least one wall (preferably two opposing walls) of the expansion channel has an angle of expansion. The angle of expansion is preferably effective to achieve a cell free layer in the expansion channel proximal to the at least one side channel and, optionally, an angle of expansion effective to produce similar cell separation at a plurality of flow rates. Preferably, the cell free layer is void of visual vortices which may trap cells near the expansion channel wall. For example, the expansion channel preferably has an angle of expansion greater than zero, such as between about 3 degrees and about 30 degrees, such as between about 5 degrees and about 20 degrees, preferably between about 5 and about 10 degrees. Preferably, the expansion channel is symmetrical along a central axis defined by fluid flow.

The tool is characterized by at least one side channel configured to remove cell-free fluid from the fluid flow. Each side channel is, therefore, located along the expansion channel and/or outlet channel where a cell free layer is located. In a preferred embodiment, the tool is characterized by two side channels located along opposing walls of the expansion channel. Preferably, each side channel is located proximal to the junction or intersection of the expansion channel and outlet channel. In embodiments and depending on the concentration of cells in the fluid and the length and volume flow of the cell-free layer, additional side channels can be added.

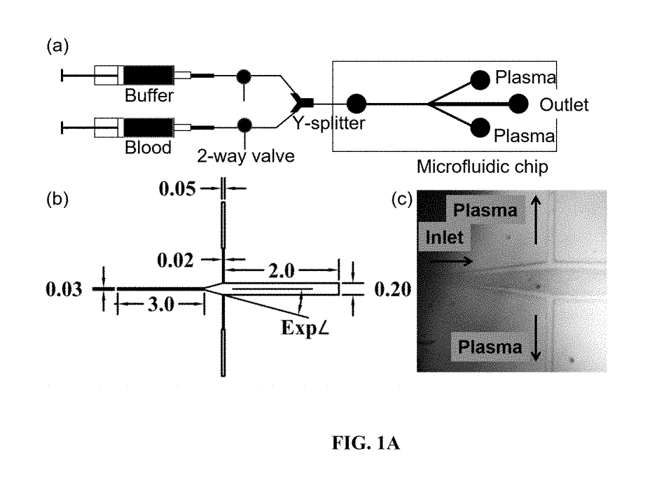

The side channels are configured to allow cell-free fluid to be removed from the cell-containing fluid. Accordingly, the side channels are preferably angled away from the fluid flow. The angle of collection can be defined by the axis along which the cell-free fluid flows at the junction of the side channel from the axis along which the remainder of the fluid flows through the device remainder of the device at this junction (e.g., the center axis in a symmetrical device). For example, the at least one side channel can be perpendicular to the fluid flow, as illustrated in view (c) of FIG. 1A. Alternatively, the angles of collection can be acute, such as illustrated in view (a) of FIG. 1A. Alternatively, the angle of collection can be obtuse, as illustrated in view (b) of FIG. 1B.

Preferably, each channel is in fluid communication with a reservoir. For example, the expansion channel can be in fluid communication with a cell collection reservoir. The side channels can be in fluid communication with cell-free fluid collection reservoirs. Where a plurality of side channels are present, the channels can communicate with the same or different reservoir. Additionally or alternatively, the inlet channel can be in fluid communication with an inlet reservoir.

A variety of materials and methods, according to certain aspects of the invention, can be used to form systems such as those described above. For example, in some embodiments, the fluid channels may comprise tubing such as, for example, flexible tubes (e.g., PEEK tubing), capillary tubes (e.g., glass capillary tubes), and the like. In some embodiments, various components can be formed from solid materials, in which microfluidic channels can be formed via micromachining, film deposition processes such as spin coating and chemical vapor deposition, laser fabrication, photolithographic techniques, etching methods including wet chemical or plasma processes, and the like. In one embodiment, at least a portion of the fluidic system is formed of silicon by etching features in a silicon chip. Technologies for precise and efficient fabrication of various fluidic systems and devices of the invention from silicon are known. In another embodiment, various components of the systems and devices of the invention can be formed of a polymer, for example, a polymer such as polydimethylsiloxane (“PDMS”), polytetrafluoroethylene (“PTFE”), or the like. In some cases, various components of the system may be formed in other materials such as metal, ceramic, glass, Pyrex®, etc.

Different components can be fabricated of different materials. For example, a base portion including a bottom wall and side walls can be fabricated from a transparent or at least partially transparent material, such as glass or a transparent polymer, for observation and/or control of the fluidic process, and a top portion can be fabricated from an opaque material such as silicon or PDMS. Components can be coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

In one embodiment, various components of the invention are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are preferred in one set of embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65 C. to about 75 C. for exposure times of, for example, about an hour. Also, silicone polymers, such as PDMS, can be elastomeric, and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures of the invention from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy, et al.), incorporated herein by reference.

In some embodiments, certain microfluidic structures of the invention (or interior, fluid-contacting surfaces) may be formed from certain oxidized silicone polymers. Such surfaces may be more hydrophilic than the surface of an elastomeric polymer. Such hydrophilic channel surfaces can thus be more easily filled and wetted with aqueous solutions.

In one embodiment, a bottom wall, or bottom or floor, of a microfluidic device of the invention is formed of a material different from one or more side walls or a top wall, or other components. For example, the interior surface of a bottom wall can comprise the surface of a silicon wafer or microchip, or other substrate. Other components can, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques can be used, as would be apparent to those of ordinary skill in the art, including, but not limited to, the use of separate adhesives, bonding, solvent bonding, ultrasonic welding, etc.

Conveniently, the inlet channel, the expansion channel, the at least one side channel, and the outlet channel are on a support or base portion, as described above. Additionally, any reservoirs can be located on the same or different support. In the examples, a silicon wafer was used.

The length of each channels can be optimized for yields and separation. Preferably, the inlet channel can be less than about 5 cm, preferably less than about 1 cm, in length, and/or the at least one side channels are less than about 5 cm, preferably less than about 1 cm. Preferably, the entire length is the tool is less than about 10 cm, thereby achieving a tool on a microfluidic chip which can be portable and/or incorporated into a handheld device.

The tool is preferably configured to cause a horizontal fluid flow and horizontal separation. The fluid flow can result from a pump or driven by hand. Where the expansion is angle is selected to result in flow rate independent separation, such as can be achieved by hand. Thus, the tool is preferably configured to receive fluid pumped by hand. An example of this can be achieved by configuring the inlet channel to be in fluid communication with a fluid inlet port adapted to receive a handheld syringe. In such an embodiment, it may be desirable to also have a second syringe that contains a buffer or wash fluid to wet the inlet channel before any cell-containing fluid is introduced into the device. The inlet port(s) can comprise tubing, valves to assist in controlling fluid flow and the like.

The tool of the invention can separate and/or concentrate fluids and cells from or in a cell-containing fluid. The cell-containing fluid can be a sample obtained from a point of care facility and can be from a human or mammalian sample. The tool is particularly useful for separating plasma from whole blood or diluted blood. As red blood cells are small, deformable, and discoid, their separation is particularly conducive to the tool. In embodiments, it may be desirable to dilute the sample, for example, with a buffer.

The output from the tool can be used for analyte testing, such as point-of-care diagnostics. For example, plasma collected from the device can be tested for the presence of malarial proteins, as described herein. Thus, the at least one side channel (or outlet channel) can be in fluid communication with an analyte test system.

Thus the invention includes a method of cell separation and/or blood plasma collection comprising pumping cell-containing fluid, such as blood into a device as described herein. The plasma so collected can preferably be at least 95%, preferably at least 99% free of cells and/or has a separation ratio of at least about 100, preferably at least about 1000, more preferably at least about 10,000. The volume of fluid which can be pumped into the tool can be less than about 100 such as less than about 50 preferably less than about 20 μL of analyte fluid. The rate of introduction is preferably effective to provide a cell-free layer proximal to the at least one side channel and/or to prevent visual vortices proximal to the at least one side channel. For example, the analyte fluid can be introduced at a rate between 10-500 μL/min, preferably between about 30-200 μL/min.

### Examples

The following examples are offered by way of illustration and are not to be construed as limiting the invention as claimed in any way.

Materials and Methods

Device Fabrication.

The separation devices were fabricated using photolithography techniques.²⁹ A 4″ silicon wafer was coated with a 100 μm thick layer of SU-8 2050 (MicroChem) and patterned using a 25400 dpi transparency mask (CAD/Art Services, Inc.). A 1 cm thick layer of PDMS (Sylgard 184, Dow Corning) was cast onto the SU-8 mold and cured for 2 hr at 80° C. The PDMS was then peeled off and the inlet and outlet holes were punched using a 1.5 mm ID×1.91 mm OD Harris Uni-Core Puncher (Ted Pella, Inc.). The device was bonded to a glass microscope slide with a thin layer of PDMS using oxygen plasma oxidation (model PDC-32G, Harrick, Ithaca, N.Y.) using 30 s exposure time on high level. Idex 1526 FEP 1/16″ OD, 0.020″ ID tubing (Upchurch Scientific) was used for inlet and outlet connections. For the inlet, Idex P-152 Y-splitter and Idex P-732 two-way valves (Upchurch Scientific) were used. The tubing was attached to luer-lock syringes (BD) using Idex P-658 female luer adapters (Upchurch Scientific). The flow was driven either using a syringe pump (Harvard Apparatus PHD 2000), or pushed by hand. The device design, shown in FIG. 1A, features 100 μm tall channels, with a 30 μm wide and 3 mm long inlet channel, which then expands at a range of angles (5-45°) to the full 200 μm wide and 2 mm long outlet channel. The plasma channels are each 20 μm wide at the skimming region, but then expand to a 50 μm width for certain designs, depending on the plasma channel resistance of interest. Typically, the 20 μm length varied from 0.5 mm to 1.3 mm in length, and the 50 μm length varied from 0 cm to 1.6 mm in length in order to get the full range of yields presented in this work.

Blood Handling.

Human blood (Research Blood Components) was collected in EDTA-coated vacutainer tubes (BD) and stored at 4° C. for up to 10 days, while the cell health was monitored visually. ACD-coated tubes (BD) produced comparable blood storage results, but were not used for the final data collection. No other anticoagulant was added. PBS (VWR) was used for blood sample dilutions. As seen in FIG. 1A, the inlet tubing is connected to both a blood and a buffer syringe through a Y-splitter. For separation reproducibility, the bubble that forms when the syringe is connected to the inlet tubing was washed away prior to experimental run. Briefly, a small amount of blood (<5 with the entrance bubble, is initially loaded into the tubing just past the Y-splitter. This entrance bubble is then washed away with the buffer connected to the Y-splitter. Buffer consists of PBS and glycerol (Sigma Aldrich); its viscosity is matched to the (diluted) plasma viscosity. Then the blood is loaded for the experimental separation. The buffer contains a colored dye in order to indicate when the blood plasma fraction is exiting the device.

Sample Analysis.

The loaded blood cell count was calculated using 1000 times diluted blood using a hemocytometer (VWR) and a microscope (Nikon Diaphot TMD). The plasma samples were collected in Eppendorf tubes (VWR), weighed out for mass fraction. If cells were visible in the plasma, the sample was diluted with PBS (VWR) until plasma had a light pink color. The cell count was calculated using a hemocytometer (VWR).

Photopolymerization-Based Colorimetric Sensing on Paper.

To prepare PfHRP2 dilutions, the appropriate PfHRP2 protein concentration was added into undiluted human blood, with volumes chosen based on the plasma volume needed for testing. The blood sample was loaded onto the device and approximately 30 μL of plasma was collected for three replicates and tested using the paper-based immunoassay with a polymerization-based colorimetric readout, according to procedures described elsewhere.²⁵ Briefly, an anti-PfHRP2 antibody (Arista Biologicals, Clone 44) was covalently coupled to aldehyde groups in the hydrophilic test zones of wax-printed, oxidized chromatography paper. 10 μL of the plasma sample collected from the separation device was applied to each test zone for 30 min. The test zones were rinsed with 1×PBS and contacted with 5 μL of 50 μg mL⁻¹ solution of anti-PfHRP2 antibody (Arista Biologicals, Clone 45) covalently conjugated to eosin, a photoinitiator. After 30 min, the surfaces were rinsed sequentially with 0.1% Tween 20 in 1×PBS, 1×PBS and de-ionized water, respectively, to remove any unbound secondary antibody. The polymerization reaction was performed by adding 20 uL of an aqueous amplification solution (pH 7.9) containing 200 mM poly(ethylene glycol) diacrylate, 150 mM triethanolamine, 100 mM 1-vinyl-2-pyrrolidinone, 0.35 μM eosin Y disodium salt, and 1.6 mM phenolphthalein to each test zone and irradiating the surface from above using an array of light-emitting diodes from an ampliPHOX® Reader (InDevR) (λ=522 nm, 30 mW cm⁻²) for 80 s. The unpolymerized aqueous solution was rinsed with water and 2 μL of 0.5 M NaOH was added to each test-zone to increase the pH for colorimetric readout. The presence of a hydrogel in the test zone is indicated by a deep pink color produced by phenolphthalein trapped in the hydrogel and is visible to the unaided eye. The images of the colorimetric readout were acquired using a cellphone and are presented without any modification. The average colorimetric intensity of each test zone was calculated from the images using ImageJ (US National Institutes of Health).

### Results and Discussion

Design Principle.

For Reynolds numbers greater than 1, when inertia dominates over viscosity, dilute rigid particles experience a balance of two forces in a straight microfluidic channel. The shear induced lift pushes particles towards the wall due to the parabolic velocity profile, while the wall-induced lift pushes particles towards the center, due to the asymmetric wake created near the wall. If the particle is deformable and non-spherical, such as a red blood cell, many other factors can locally dominate the flow profile, especially when the cell suspension is dense, such as in human blood.¹⁴⁻¹⁷ Experimentally, it has been observed that this behavior leads to a high number of cells near the center of the channel, thus creating a cell-free layer near the channel walls. This cell-free layer can be further improved upon by passing fluid through a constricted, small channel before collecting in a larger, expanded channel location. By studying a range of expansion regimes, as defined in FIG. 1A, we were able to improve separation values as well as ensure result reproducibility. Initial experiments focused on a range of expansion angles, 90° and 5-45° in increments of 5°. For the chosen channel dimensions and flow rates of interest, all expansion angles larger than 20° produced visible vortices, which trapped red blood cells, and generated large variations in the separation results.

The most gradual expansion, at 5°, produced stable flow profiles at a range of flow rates, from 30-200 μL/min with no visual vortices. Ideally, a channel expansion should temporarily increase the cell free layer while minimizing the number of red blood cells near the expansion region. The optimal expansion angle could vary for different blood dilutions and plasma yields due to changes in viscosity and different expansion flow profiles. Due to blood complexity and high density suspension of cells, we chose to experimentally study the full 5-20° range of expansion angles in order to better understand blood behavior in expansion regions. Of note, this separation design is very sensitive to pressure drops across each channel, and therefore can experience disturbance due to bubble or cell deposition on the channel surface. In order to improve consistency of blood loading, a prewetting buffer step was found essential for separation reproducibility.

Constriction-Expansion Separation Results.

The diluent to blood ratio was defined as blood volume fraction=V_{blood}/Vₜₒₜₐₗ, where V_{blood} is the blood volume and Vₜₒₜₐₗ is the total solution volume. Therefore, for 10 times diluted blood the blood volume fraction is 0.1×, and undiluted whole blood is defined as 1× blood. The expansion behavior was further studied experimentally for 0.1×, 0.33× and 1× human blood in 5°, 10°, 15°, and 20° expansion designs. The resistance between the outlet and plasma collection channels was varied through channel-defined resistance as well as external tubing resistance, in order to give a range of mass fractions and separation values. Mass fraction or yield is defined as the mass of the isolated plasma collected, over the total mass that was injected into the device, Yield=massᵢₛₒₗₐₜₑ/massₜₒₜₐₗ. Therefore, the maximum yield that is achievable is between 0.4-0.6, depending on the plasma fraction in whole blood, which can range between approximately 40-60%. Plasma purity, which is commonly used to characterize these systems, is defined as Purity=100%(1−Cᵢₛₒₗₐₜₑ/Cᵢₙₗₑₜ). Given that there are approximately 10⁹ cells in human blood, even if purity is defined at 99%, there are still on the order of 10⁷ cells in a milliliter of isolate collected. Thus, purity is not a useful metric for characterizing remaining cells in plasma.

In order to characterize our system beyond the metrics commonly used in the blood separation literature, we defined an isolate separation ratio, SepRatio=Cᵢₙₗₑₜ/Cᵢₛₒₗₐₜₑ, where Cᵢₙₗₑₜ and Cᵢₛₒₗₐₜₑ is the number of red blood cells per mL in the inlet and the plasma isolate, respectively. This term allows us to increase the blood plasma separation variable space, where 99% purity correlates to an isolate separation ratio of 100. And therefore any ratios above this value explore a new passive blood separation space. An ideal system would collect a large amount of plasma (high yield) with a very small amount of cells in the isolated plasma (high SepRatio).

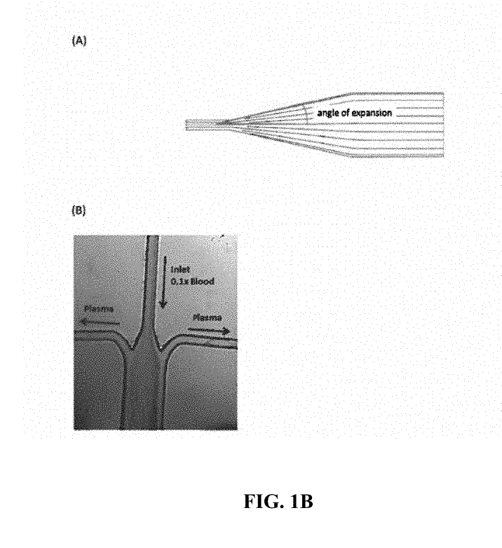

The initial design study tested 5°-45° expansions using 0.1× blood, at flow rates of 50-200 μL/min, as defined by a syringe pump. As discussed previously, the 20° and higher expansions did not give us predictable plasma mass fractions or SepRatio, due to possible vortices at these angles. 5°, 10°, and 15° expansion designs gave comparable separation results across a range of mass fractions, resulting in 1,000-50,000 separation ratios for 15-5% yield, respectively, as seen in FIG. 2. By introducing the SepRatio parameter, we are able to study data beyond the 99% purity parameter, which is marked on the plot for reference. Similar results were observed for 0.33× blood plasma separation, as for 0.1× blood.

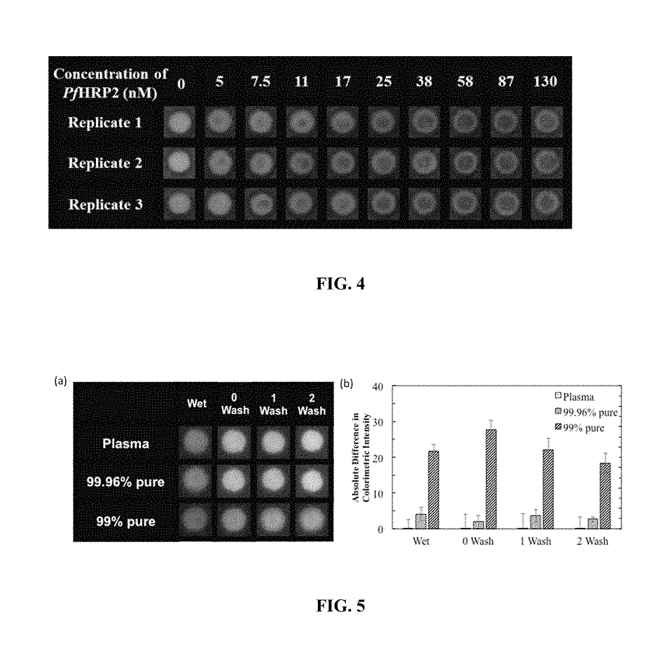

When undiluted blood is used, the purity and isolate separation ratio for the 10° and 15° expansion angles drop off drastically, as seen in FIG. 2. Interestingly, the separation ratio and yield are largely conserved for the 5° expansion angle even when using undiluted whole blood. The 5° expansion angle resulted in separation ratio of 2,800 with corresponding yield of 8%; this constitutes a twenty-eight-fold improvement over what has currently been demonstrated in the literature, as seen in Table 1. By significantly decreasing the cell concentration in the isolate sample, we reduce the probability of high background or even sensor fouling, e.g. by visual comparison of 100 versus 2,800 or more isolate separation ratio for undiluted whole blood. A 100 separation ratio sample has a clearly visible concentration of red blood cells present leading to an overall pink color, while a 2,800 separation ratio does not have visible red blood cells present and is much closer in color to the pure plasma sample. Therefore, increasing the separation ratio beyond the 99% purity that's been demonstrated previously in literature can have a significant impact on the background signal in colorimetric (FIG. 5) and other visual sensors.

TABLE 1
Comparison of plasma purity and yield for undiluted whole blood.

| Authors | Purity | SepRatio | Yield |
|---|---|---|---|
| Jaggi, R. et al. (pump)²² | 31% | 1.4 | 0.04 |
| Tripathi, S. et al. (pump)²³ | 67% | 3.0 | 0.018 |
| Kersaudy-Kerhoas, M. et al. (pump)¹⁸ | 99% | 100 | 0.05 |
| 5° expansion design (pump) | 99.96% | 2800 | 0.08 |
| 5° expansion design (hand) | 99.96% | 2800 | 0.09 |

Given that the mean red cell volume is 80-100 fL, the hematocrit of the whole blood samples used was approximated to vary between 40-50% based on the hemocytometer cell count. There was no dependence of the separation ratio on the hematocrit in any of the expansion designs studied. This could be due to the small plasma fraction collected when compared to the total plasma available (50-60% of total volume for undiluted blood and even larger values for diluted blood). Since all of the channel parameters were larger than the size of cells present in the blood, clogging was not an issue and device lifetime allowed for the processing of around 300 mL of blood before any build-up that would alter the device flow profile was noticeable.

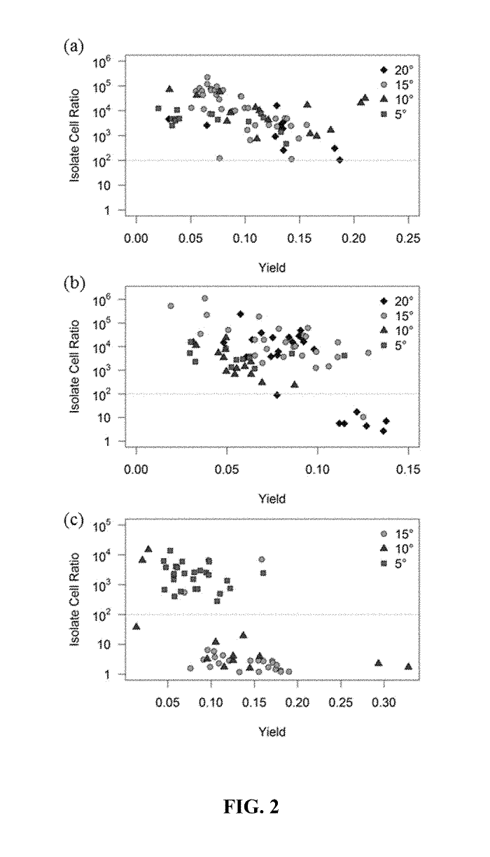

The 5° expansion design, which did not show sensitivity to flow rate and produced significant separation for undiluted blood, was further studied for portability applications. Instead of a syringe pump setup, the blood was injected into the device using a syringe pushed steadily by hand. The flow rate was approximated to be 50 and 200 μL/min for the undiluted and diluted blood, respectively. The separation ratio and mass fraction values are presented in FIG. 3. The separation value, 2,800, is the same as the syringe pump setup for a 9% yield. These results demonstrate of a simple, portable design that separates out the plasma fraction and can be further incorporated with other microfluidic constructs. In order to ensure minimal cell-damage was incurred to the blood in the device, the plasma color was visually evaluated after every experiment.³⁰ The collected fractions were also monitored under the microscope and showed no ruptured red blood cells. The plasma at the highest flow rates were also checked for hemoglobin release using the Cripps Method and showed minimal lysing, comparable to standard centrifuging procedures.³¹⁻³³

TABLE 2
Mean percent lysis for red blood cells using three different methods
for second day old undiluted whole blood. Decanting separation
involved direct plasma removal (using a pipettor) from a BD
Vacutainer tube (Becton Dickinson) that was stored overnight in a
refrigerator in order to allow for cell settling and plasma decanting.
Centrifuge separation (laboratory standard) involved 1.5 mL
Eppendorf LoBind tubes (Sigma Aldrich) that were spun down at 1 g
for 10 min before plasma was removed from the top using a pipette.
Device separation was done for undiluted blood on a 5° expansion
device for 50-300 μL/min flow rates, which led to similar low lysing
values. The % red blood cell lysed was calculated using the
Cripps Method.

| | Av | 1 Std Dev of |
|---|---|---|
| Method | % Lysis | % Lysis |
| Decanting | 0.059% | 0.006% |
| Centrifuge | 0.095% | 0.04% |
| Device | 0.11% | 0.02% |

Using the Plasma Fraction in a Downstream Diagnostic Test for Malaria.

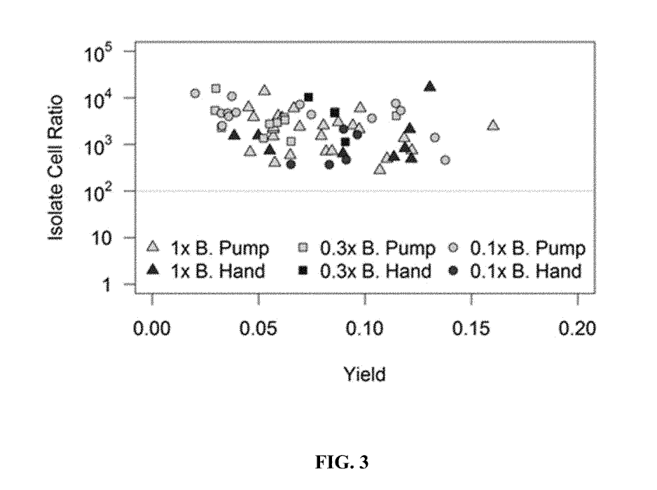

A paper-based immunoassay was used for detection of PfHRP2 in whole human blood. Nine different whole blood solutions containing PfHRP2 concentrations ranging from 5 nM to 130 nM were prepared. Plasma, containing PfHRP2, was separated for each of the nine solutions using the 5° expansion design. Whole blood that did not contain PfHRP2 served as a negative control, and plasma separated from these samples was tested along with the PfHRP2 samples using paper-based sensors. Colorimetric responses were generated using PBA. The visual limit of detection (LOD), defined as the concentration for which all replicates produced a colored readout that is visible unambiguously to the unaided eye, was found to be 7.5 nM (FIG. 4). This visual LOD value is comparable to the visual LOD value of 7.2 nM published previously²⁵ for PfHRP2 dissolved in buffer and is well below 28 nM, which was reported as the mean concentrations of PfHRP2 in plasma samples in a patient cohort with falciparum malaria.³⁴ Additional quantification of results from FIG. 4 is included in Table 3: below.

TABLE 3
Concentration of PƒHRP2 vs. p-value for the 1-tail unpaired t-test.
P-value of less than 0.05 shows statistically significant difference
between color intensity and background. Concentration of 7.5 nM and
above was found to be statistically above the background value.

| Concentration | Average | Std. Dev. of | |
|---|---|---|---|
| of PƒHRP2 | Colorimetric | Colorimetric | P-value vs. |
| (nM) | Intensity | Intensity | Background |
| 0 | 13.60 | 2.02 | |
| 5 | 14.14 | 7.26 | 0.4554 |
| 7.5 | 27.24 | 8.09 | 0.0461 |
| 11 | 31.04 | 5.18 | 0.0088 |
| 17 | 30.91 | 8.32 | 0.0309 |
| 25 | 39.78 | 5.33 | 0.0036 |
| 38 | 44.57 | 7.62 | 0.0073 |
| 58 | 53.97 | 5.37 | 0.0012 |
| 87 | 49.02 | 2.91 | 0.0001 |
| 130 | 45.39 | 11.96 | 0.0204 |

PBA is a signal amplification and detection method where binding events, such as antigen-antibody binding in an immunoassay, are used to localize a photoinitiator with an analyte of interest by covalently attaching the photoinitiator to one of the binding molecules used in the assay. When the photoinitiator present on the surface is irradiated with an appropriate dose of light in the presence of monomers, it can initiate a free-radical polymerization reaction, resulting in the formation of a hydrogel that can be seen with unaided eye. Since oxygen is a common inhibitor of the free-radical polymerization reactions, such reactions are typically carried out under purged, oxygen-free environment.³⁵⁻³⁷ However, the eosin/tertiary amine based initiation system used above has been engineered to overcome oxygen inhibition and can be carried out in air.²⁷ To produce the data in FIG. 4, eosin was localized on the surface when PfHRP2 is present on the surface by covalently conjugating eosin to the anti-PfHRP2 reporter antibody. The high-contrast colorimetric results are produced using a pH indicator, phenolphthalein, that is a component of the amplification solution and becomes entrapped in the hydrogel during polymerization.²⁵ The uncertain polymerization outcome at concentrations just below the LOD, i.e. 5 nM, can be attributed to the threshold nature of polymerization³⁵ where slight differences in the number of bound proteins can shift the photoinitiator density on the surface either above or below the concentration necessary for the propagation reactions during free-radical polymerization to compete with the inhibition reactions involving oxygen.³⁸

The results in FIG. 4 show that PfHRP2 present in whole blood samples is present in the plasma obtained from the separation device. The plasma is pure enough for the PBA-based test such that any remaining red blood cells or components of blood that are either eliminated or not produced during traditional, laboratory-based preparation of serum do not interfere with the free radical polymerization reaction. Species of potential concern due to their participation in radical reactions include hemoglobin and other iron-containing compounds. The plasma samples obtained from our device were visually and quantifiably similar to the plasma samples obtained via centrifugation and did not produce any background color on paper that could interfere with interpretation of the colorimetric PBA readout (FIG. 5). Additionally, the visual LoD in plasma samples produced from the separation device is similar to the visual LoD obtained in buffer samples and is well below the clinically relevant concentration range of PfHRP2. These results are the first demonstration where PBA has been used to detect an analyte directly from whole blood samples by pairing a low-cost paper-based colorimetric test with a portable blood-plasma separation device. The hand-operated plasma separation device can therefore provide plasma samples from small volumes of whole blood that can be successfully used with colorimetric tests and make these tests feasible outside the laboratory infrastructure, in low-resource settings where they are needed the most.

### CONCLUSIONS

In this work, we developed a portable, blood plasma separation design that further improves the plasma skimming separation approach with the incorporation of a gradual expansion region. For undiluted blood, the optimal 5° expansion gave reproducible separation ratio of approximately 2800 for 9% yield, when driven by hand. Similar separation ratios and yields were observed for a range of expansion angles, 5-15°, when 3 and 10 times diluted blood was used. The chosen channel dimensions and the simple design minimized clogging and produced a plasma throughput of 5-30 μL/min for undiluted and diluted blood, respectively. The system was used to produce samples for analysis using paper-based sensors of PfHRP2, a biomarker of malaria, resulting in colorimetric sensing with the unaided eye down to 7.5 nM in whole blood, which is well below the clinically relevant value of 28 nM.³⁴ The constriction-expansion design provides an improvement in undiluted blood separation for passive microfluidic methods, and could be a future resource for experimental and theoretical studies of the formation of the cell free layer in channels on the micrometer scale.

The following references are incorporated herein by reference in their entirety.

### REFERENCES

(1) Yu, Z. T. F.; Aw Yong, K. M.; Fu, J. Small 2014, 10, 1687-1703.
(2) Kersaudy-Kerhoas, M.; Sollier, E. Lab on a Chip 2013, 13, 3323-3346.
(3) Tajudin, A. A.; Petersson, K.; Lenshof, A.; Sward-Nilsson, A. M.; Aberg, L.; Marko-Varga, G.; Malm, J.; Lilja, H.; Laurell, T. Lab on a Chip 2013, 13, 1790-1796.
(4) Aran, K.; Fok, A.; Sasso, L. A.; Kamdar, N.; Guan, Y.; Sun, Q.; Undar, A.; Zahn, J. D. Lab on a Chip 2011, 11, 2858-2868.
(5) Kumar, A. A.; Hennek, J. W.; Smith, B. S.; Kumar, S.; Beattie, P.; Jain, S.; Rolland, J. P.; Stossel, T. P.; Chunda-Liyoka, C.; Whitesides, G. M. Angewandte Chemie International Edition 2015, 54, 5836-5853.
(6) Geng, Z.; Ju, Y; Wang, Q.; Wang, W.; Li, Z. RSC Advances 2013, 3, 14798-14806.
(7) Brigden, M. L. American family physician 1999, 60, 1443-1450.
(8) Tachi, T.; Kaji, N.; Tokeshi, M.; Baba, Y. Analytical Chemistry 2009, 81, 3194-3198.
(9) Wang, S.; Sarenac, D.; Chen, M. H.; Huang, S.-H.; Giguel, F. F.; Kuritzkes, D. R.; Demirci, U. International journal of nanomedicine 2012, 7, 5019.
(10) Crowley, T. A.; Pizziconi, V. Lab on a Chip 2005, 5, 922-929.
(11) Son, J. H.; Lee, S. H.; Hong, S.; Park, S.-m.; Lee, J.; Dickey, A. M.; Lee, L. P. Lab on a Chip 2014, 14, 2287-2292.
(12) VanDelinder, V; Groisman, A. Analytical Chemistry 2006, 78, 3765-3771.
(13) Choi, S.; Song, S.; Choi, C.; Park, J.-K. Small 2008, 4, 634-641.
(14) Park, J.-S.; Song, S.-H.; Jung, H.-I. Lab on a Chip 2009, 9, 939-948.
(15) Di Carlo, D.; Irimia, D.; Tompkins, R. G.; Toner, M. Proceedings of the National Academy of Sciences 2007, 104, 18892-18897.
(16) Amini, H.; Lee, W.; Di Carlo, D. Lab on a Chip 2014, 14, 2739-2761.
(17) Geislinger, T. M.; Franke, T. Advances in Colloid and Interface Science 2014, 208, 161-176.
(18) Kersaudy-Kerhoas, M.; Kavanagh, D. M.; Dhariwal, R. S.; Campbell, C. J.; Desmulliez, M. P. Y. Lab on a Chip 2010, 10, 1587-1595.
(19) Wang, J.; Ahmad, H.; Ma, C.; Shi, Q.; Vermesh, O.; Vermesh, U.; Heath, J. Lab on a Chip 2010, 10, 3157-3162.
(20) Doyeux, V.; Podgorski, T.; Peponas, S.; Ismail, M.; Coupier, G. Journal of Fluid Mechanics 2011, 674, 359-388.
(21) Yang, S.; Undar, A.; Zahn, J. D. Lab on a Chip 2006, 6, 871-880.
(22) Jaggi, R.; Sandoz, R.; Effenhauser, C. Microfluidics and Nanofluidics 2007, 3, 47-53.
(23) Tripathi, S.; Prabhakar, A.; Kumar, N.; Singh, S.; Agrawal, A. Biomed Microdevices 2013, 15, 415-425.
(24) Fan, R.; Vermesh, O.; Srivastava, A.; Yen, B. K. H.; Qin, L.; Ahmad, H.; Kwong, G. A.; Liu, C.-C.; Gould, J.; Hood, L.; Heath, J. R. Nat Biotech 2008, 26, 1373-1378.
(25) Badu-Tawiah, A. K.; Lathwal, S.; Kaastrup, K.; Al-Sayah, M.; Christodouleas, D. C.; Smith, B. S.; Whitesides, G. M.; Sikes, H. D. Lab on a Chip 2015, 15, 655-659.
(26) World Health Organization, Malaria Rapid Diagnostic Test Performance—Results of WHO product testing of malaria RDTs: Round 4 (2012) 2012.
(27) Kaastrup, K.; Sikes, H. D. Lab on a Chip 2012, 12, 4055-4058.
(28) Lathwal, S.; Sikes, H. D. Lab on a Chip 2016, 16, 1374-1382.
(29) Duffy, D. C.; McDonald, J. C.; Schueller, O. J. A.; Whitesides, G. M. Analytical Chemistry 1998, 70, 4974-4984.
(30) Sowemimo-Coker, S. O. Transfusion medicine reviews 2002, 16, 46-60.
(31) Cripps, C. M. Journal of Clinical Pathology 1968, 21, 110-112.
(32) Malinauskas, R. A. Artificial Organs 1997, 21, 1255-1267.
(33) Han, V.; Serrano, K.; Devine, D. V. Vox Sanguinis 2010, 98, 116-123.
(34) Dondorp, A. M.; Desakorn, V; Pongtavornpinyo, W.; Sahassananda, D.; Silamut, K.; Chotivanich, K.; Newton, P. N.; Pitisuttithum, P.; Smithyman, A. M.; White, N. J.; Day, N. P. J. PLoS Med 2005, 2, e204.
(35) Sikes, H. D.; Hansen, R. R.; Johnson, L. M.; Jenison, R.; Birks, J. W.; Rowlen, K. L.; Bowman, C. N. Nat Mater 2008, 7, 52-56.
(36) Hansen, N. M. L.; Plackett, D. Biomacromolecules 2008, 9, 1493-1505.
(37) Kuck, L. R.; Taylor, A. W. BioTechniques 2008, 45, 179-186.
(38) Kaastrup, K.; Chan, L.; Sikes, H. D. Analytical Chemistry 2013, 85, 8055-8060.

While this invention has been particularly shown and described with references to preferred embodiments thereof, it will be understood by those skilled in the art that various changes in form and details may be made therein without departing from the scope of the invention encompassed by the appended claims.

1. A cell separation tool comprising:
an inlet channel;
an expansion channel in fluid communication with the inlet channel, a width of a proximal end of the expansion channel being approximately equal to a width of the inlet channel, and a width of a distal end of the expansion channel being larger than the width of the inlet channel;
at least one side channel in fluid communication with the expansion channel and configured to divert fluid flow from the expansion channel; and
an outlet channel in fluid communication with the expansion channel.

2. The tool of claim 1, wherein the outlet channel has a width equal to the distal end of the expansion channel.

3. The tool of claim 1, wherein the expansion channel has an angle of expansion effective to achieve a cell free layer in the expansion channel proximal to the at least one side channel and, optionally, an angle of expansion effective to produce similar cell separation at a plurality of flow rates.

4. The tool of claim 1, wherein the expansion channel has an angle of expansion between about 5 degrees and about 20 degrees, preferably between about 5 and 10 degrees.

5. The tool of claim 1, wherein the width of the proximal end of the expansion channel being approximately equal to the width of the inlet channel is a horizontal width, and the width of the distal end of the expansion channel being larger than the width of the inlet channel is a horizontal width.

6. The tool of claim 1, wherein the width of the at least one side channel at its point of communication with the expansion channel is less than or equal to the width of the inlet channel.

7. The tool of claim 1, wherein each channel is a microchannel.

8. The tool of claim 1, wherein the width of the inlet channel is less than about 100 μm, and/or the width of the outlet channel is less than about 200 μm, and/or the width of the at least one side channel is less than about 100 μm.

9. The tool of claim 1, wherein the height of the inlet channel, the expansion channel, the at least one side channel and the outlet channel are each, independently, less than about 200 μm.

10. The tool claim 1, wherein the expansion channel and each side channel are in fluid communication with a collection reservoir.

11. The tool of claim 1, wherein the inlet channel is in fluid communication with an inlet reservoir.

12. The tool of claim 1, wherein the inlet channel, the expansion channel, the at least one side channel, and the outlet channel are on a support.

13. The tool of claim 12, wherein the support is a glass or silicon wafer.

14. The tool of claim 1, wherein the inlet channel is less than about 1 cm in length, and/or the at least one side channels are less than 1 cm.

15. The tool of claim 1, wherein the at least one side channel is angled away from the fluid flow.

16. The tool of claim 1, wherein the at least one side channel is perpendicular to the fluid flow.

17. The tool of claim 1, wherein the at least one side channel is perpendicular to the inlet channel and the outlet channel.

18. The tool of claim 1, wherein the tool is configured to cause a horizontal fluid flow and horizontal separation.

19. The tool of claim 1, wherein the inlet channel and expansion channel are symmetrical along a central axis and the at least one side channel is perpendicular to the central axis.

20. The tool of claim 1, the at least one side channel is located at the junction of the expansion channel and outlet channel.

21. The tool of claim 1, wherein at least one side channel is located along each wall of the expansion channel.

22. The tool of claim 1, wherein two side channels are located at the junction of the expansion channel and outlet channel.

23. The tool of claim 1, wherein the inlet channel has a first fluid flow region and the expansion channel has a second flow region with a different fluid flow pattern than the first fluid flow region.

24. The tool of claim 1, wherein the tool is configured to receive fluid pumped by hand.

25. The tool of claim 1, wherein the inlet channel is in fluid communication with a fluid inlet port adapted to receive a handheld syringe.

26. The tool of claim 1, wherein the at least one side channel is in fluid communication with an analyte test system.

27. The tool of claim 26, wherein the analyte test system detects an analyte in blood plasma.

28. A blood plasma collection tool comprising:
an inlet channel;
an expansion channel in fluid communication with the inlet channel, a width of the expansion channel expanding from a proximal end of the expansion channel to a distal end of the expansion channel at an angle greater than 0 degrees;
at least one side channel in fluid communication with the expansion channel and configured to divert fluid flow from the expansion channel; and
an outlet channel in fluid communication with the expansion channel and having a width equal to the distal end of the expansion channel.

29-36. (canceled)

37. A blood plasma collection tool comprising:
an inlet channel;
an expansion channel in fluid communication with the inlet channel, a width of a proximal end of the expansion channel being approximately equal to a width of the inlet channel, and a width of a distal end of the expansion channel being larger than the width of the inlet channel;
at least one side channel in fluid communication with the expansion channel and configured to divert fluid flow from the expansion channel; and
an outlet channel in fluid communication with the expansion channel and having a width equal to the distal end of the expansion channel.

38. A method of blood plasma collection comprising:
pumping blood into an expansion channel having a width expanding from a proximal end of the expansion channel to a distal end of the expansion channel at an angle greater than 0 degrees; and
collecting blood plasma from at least one side channel in fluid communication with the expansion channel and configured to divert fluid flow from the expansion channel.

39-46. (canceled)

47. A method for separating cells from an analyte fluid comprising:
a. Providing a tool according to claim 1;
b. Providing an analyte fluid comprising cells;
c. Introducing the fluid into the inlet channel;
d. Collecting a cell-free fluid from the at least one side channel;
e. Collecting a concentrated fluid comprising cells from the outlet channel.

48-60. (canceled)
